(19) United States
(12) Patent Application Publication
(10) Pub. No.: US 2019/0117746 A1
(43) Pub. Date: Apr. 25, 2019

(54) COMPOSITIONS, SYSTEMS, AND METHODS FOR SCAR TISSUE MODIFICATION

(71) Applicant: University of Florida Research Foundation, Inc., Gainesville, FL (US)

(72) Inventors: MARK B. SHERWOOD, GAINESVILLE, FL (US); DANIEL J. GIBSON, GAINESVILLE, FL (US); GREGORY SCOTT SCHULTZ, GAINESVILLE, FL (US)

(21) Appl. No.: 16/094,710

(22) PCT Filed: Apr. 17, 2017
(86) PCT No.: PCT/US17/27906

§ 371 Date: Oct. 18, 2018

Related U.S. Application Data
(60) Provisional application No. 62/323,921, filed on Apr. 18, 2016.

(51) Int. Cl. A61K 38/48; A61K 38/47; A61K 41/00; A61K 38/54; A61K 47/69; A61K 49/18; A61K 9/00; A61P 17/02; A61P 27/02

(52) U.S. Cl. CPC A61K 38/4886; A61F 9/00781; A61K 41/00; A61K 38/54; A61K 47/6923; A61K 47/6929; A61K 49/1866; A61K 9/0051; A61P 17/02; A61P 27/02; C12Y 302/01035; C12Y 304/24007; C12Y 304/2404; C12Y 304/21062; A61K 38/47

(57) ABSTRACT

Described herein are formulations that can include one or more enzymes that can break down one or more components of scar tissue. Also provided herein are methods of treating scar tissue by administering a formulation provided herein to a subject in need thereof.

### CROSS-REFERENCE TO RELATED APPLICATIONS

This application claims the benefit of and priority to co-pending U.S. Provisional Patent Application No. 62/323,921, filed on Apr. 18, 2016, entitled “COMPOSITIONS, SYSTEMS, AND METHODS FOR SCAR TISSUE MODIFICATION,” the contents of which is incorporated by reference herein in its entirety.

### BACKGROUND

Scar tissue is formed as part of the normal healing process of damaged tissue. The initial production of granulation during initial scar tissue formation is necessary to provide tensile strength to the injury site. However, this granulation can lead to contraction of the fibrous scar and to poor structural organization of components of the regenerating tissue. Further, scar tissue can lead to complications, such as adhesions, loss of flexibility, unsightly blemishes, pain, and in treatments where implants are employed, implant dysfunction and other complications. As such, there exists a need for improved methods of controlling and/or modifying scar tissue.

### SUMMARY

In some aspects, described herein are compositions and formulations thereof that can be used for treating excessive bleb formation in a subject, where the compositions and formulations thereof can contain an enzyme in an amount effective to form pores in a bleb, where the bleb is formed as the result of a surgical procedure to treat glaucoma in the subject. The surgical procedure can be implantation of an ocular drainage device. The surgical procedure can be a trabeculectomy. The enzyme can be coupled to a magnetic nanoparticle. The enzyme can be selected from the group of: collagenase, hyaluronidase, serrapeptase, nattokinase, FAS-ligand and combinations thereof. The formulation can be formulated for ocular injection.

Also provided herein are compositions and formulations thereof for treating scar tissue in a subject the can contain a biomolecule or an agent in an amount effective to form pores in the scar tissue or reduce an amount of scar tissue. The agent can be an enzyme. The agent can be selected from the group of: collagenase, hyaluronidase, serrapeptase, nattokinase, FAS-ligand, and combinations thereof. The agent can be coupled to a magnetic nanoparticle.

In some aspects, also described herein are methods of treating a scar tissue in a subject that can contain at least the step of delivering an agent or formulation thereof to the scar tissue or a region proximate to the scar tissue. The agent can be an enzyme. The agent can beselected from the group of: collagenase, hyaluronidase, serrapeptase, nattokinase, FAS-ligand and combinations thereof. The agent can be coupled to a magnetic nanoparticle. The methods can further include the step of exposing the scar tissue to a magnetic field. The subject can be a human. The subject can be a non-human animal. The scar tissue can be a bleb. The methods can further include the step of co-delivering a dye with the enzyme or formulation thereof. The methods can further include the step of imaging the subject using resonance imaging (MRI), computed tomography (CT), positron emission tomography (PET) scan, X-ray, or confocal microscopy.

### BRIEF DESCRIPTION OF THE DRAWINGS

Further aspects of the present disclosure will be readily appreciated upon review of the detailed description of its various embodiments, described below, when taken in conjunction with the accompanying drawings.

FIG. 1 shows a perspective view of an eye immediately after having a drainage implant placed in the sub-Tenon's capsule region of the eye between rectus muscles.

FIG. 2 shows a lateral view of the eye of FIG. 1 showing a stretched Tenon's Capsule over an endplate of the drainage implant.

FIG. 3 shows a perspective view of the eye of FIG. 1 at about two to three weeks post implant placement demonstrating initial distension of a bleb over a portion of the endplate.

FIG. 4 shows a lateral view of the eye of FIG. 3 showing bleb distension over a portion of the endplate.

FIG. 5 shows a perspective view of the eye of FIG. 1 at greater than about 3 weeks post implant placement demonstrating bleb formation around the endplate.

FIG. 6 shows a lateral view of the eye of FIG. 5 demonstrating bleb formation around the endplate.

FIG. 7 shows a perspective view of an eye with a bleb or excessive bleb and shows delivery of a formulation as described herein directly into the encapsulated bleb space (needle position A) between the endplate and the bleb/Tenon's capsule tissue and shows delivery of a formulation as described herein into the sub-conjunctival space and to the space present at the outer surface of the bleb (needle position B).

FIG. 8 shows a lateral view of an eye with a bleb or excessive bleb with magnetic enzyme nanoparticles present in the aqueous space between the endplate and the bleb/Tenon's capsule tissue prior to exposure to a magnetic field.

FIG. 9 shows a lateral view of an eye with a bleb or excessive bleb with magnetic enzyme nanoparticles present in the aqueous space between the endplate and the bleb/Tenon's capsule tissue during exposure to a magnetic field.

FIGS. 10A-10B show images of identifying implant locations in an in vivo rat skin model at 6-10 weeks post-implant insertion.

FIG. 11 shows an image of performing injection of a formulation into a model bleb in the in vitro rat skin model shown and described in association with FIGS. 10A and 10B. Needle position A demonstrated in FIG. 7 was used for composition delivery.

FIG. 12 shows a graph demonstrating permeability of the rat capsule after delivery of various formulations.

FIG. 13A shows an image of a control capsule matured for about 10 weeks that were injected with saline.

FIG. 13B shows a representative image of a pre-placed tube attached to the implant, exiting through the skin and connected to the pump and pressure detector system (10 weeks post GDD implantation)

FIGS. 14A-14C show images of capsules matured for about 6 weeks (FIG. 14A) or 10 weeks (FIGS. 14B and 14C) injected with 0.01 mg of FAS ligand.

FIG. 15 shows an image of a capsule that was matured for about 6 weeks that was injected with 138 collagenase destructive units (CDU).

FIGS. 16A-16B shows images of a capsule matured for about 10 weeks that was injected with 12.5 CDU before capsule tear (FIG. 16A) and after capsule tear (FIG. 16B).

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed. As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of molecular biology, microbiology, nanotechnology, organic chemistry, biochemistry, botany, physiology, ophthalmology, optometry, medical and veterinary science, and the like, which are within the skill of the art. Such techniques are explained fully in the literature.

### Definitions

As used herein, “about,” “approximately,” and the like, when used in connection with a numerical variable, generally refers to the value of the variable and to all values of the variable that are within the experimental error (e.g., within the 95% confidence interval for the mean) or within ±10% of the indicated value, whichever is greater.

As used herein, “control” is an alternative subject or sample used in an experiment for comparison purposes and included to minimize or distinguish the effect of variables other than an independent variable. A “control” can be positive or negative.

As used herein, “effective amount” refers to the amount of a composition or pharmaceutical formulation described herein that will elicit a desired biological or medical response of a tissue, system, animal, plant, protozoan, bacteria, yeast or human that is being sought by the researcher, veterinarian, medical doctor or other clinician. The effective amount will vary depending on the exact chemical structure of the composition or pharmaceutical formulation, the causative agent and/or severity of the infection, disease, disorder, syndrome, or symptom thereof being treated or prevented, the route of administration, the time of administration, the rate of excretion, the drug combination, the judgment of the treating physician, the dosage form, and the age, weight, general health, sex and/or diet of the subject to be treated. “Effective amount” can refer to an amount of a composition or pharmaceutical formulation described herein can be the amount effective to decrease the amount of scar tissue, decreasing the amount of bleb tissue, decreasing the amount of fluid in a bleb, and/or dissolve scar (e.g. bleb) tissue.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “pharmaceutically acceptable salt” refers to any acid or base addition salt whose counter-ions are non-toxic to the subject to which they are administered in pharmaceutical doses of the salts.

As used interchangeably herein, “subject,” “individual,” or “patient,” refers to a vertebrate, preferably a mammal, more preferably a human. Mammals include, but are not limited to, murines, simians, humans, farm animals, sport animals, and pets. The term “pet” includes a dog, cat, guinea pig, mouse, rat, rabbit, ferret, and the like. The term farm animal includes a horse, sheep, goat, chicken, pig, cow, donkey, llama, alpaca, turkey, and the like.

As used herein, “active agent” or “active ingredient” refers to a component or components of a composition to which the whole or part of the effect of the composition is attributed.

As used herein, “dose,” “unit dose,” or “dosage” refers to physically discrete units suitable for use in a subject, each unit containing a predetermined quantity of the nanoparticle composition or formulation calculated to produce the desired response or responses in association with its administration.

As used herein “immunomodulator,” refers to an agent, such as a therapeutic agent, which is capable of modulating or regulating one or more immune function or response.

As used herein “anti-infectives” can include, but are not limited to, antibiotics, antibacterials, antifungals, antivirals, and antiproatozoals.

As used herein, “therapeutic” refers to treating, healing, and/or ameliorating a disease, disorder, condition, or side effect, or to decreasing in the rate of advancement of a disease, disorder, condition, or side effect. The term also includes within its scope enhancing normal physiological function, palliative treatment, and partial remediation of a disease, disorder, condition, side effect, or symptom thereof. The term “therapeutic” can refer to reducing the amount and/or size of scar tissue (e.g. bleb).

As used herein, “preventative” and “prevent” refers to hindering or stopping a disease or condition before it occurs, even if undiagnosed, or while the disease or condition is still in the sub-clinical phase.

### Discussion

Glaucoma refers to a group of eye diseases that can result in irreversible blindness. In most cases, glaucoma produces an increase in intraocular pressure as compared to what is normal for that individual. The increased pressure can damage the optic nerve, which results in irreversible eyesight loss. Though there is no cure for glaucoma there are therapies available, which include, surgical management and drug therapies. Surgical management options include trabeculectomy and implantation of drainage devices that provide outlets for fluid drainage from the eye to relieve intraocular pressure.

The use of glaucoma drainage implants has steadily increased over the last 40 years, especially relative to other surgical glaucoma procedures, such as trabeculectomy. All modern glaucoma drainage implants contain a tube that shunts aqueous humor from the anterior eye space to an endplate (also called an explant) that can be surgically inserted in the equatorial region of the globe. The drainage implants can be valved or unvalved.

Following implantation of a glaucoma drainage device, a fibrous capsule forms around the end plate over a period of several weeks. This fibrous capsule is also referred to as a “bleb” and is essentially scar tissue. Aqueous humor pools in the potential space between the end plate and the surrounding, non-adherent fibrous capsule when flow occurs through the anterior chamber tube. Aqueous humor then passes through the capsule via the process of passive diffusion and is absorbed by periocular capillaries and lymphatics. The fibrous capsule around the endplate is the source of major resistance to the aqueous flow with drainage implants. The degree of intraocular pressure reduction observed following glaucoma drainage implant surgery is dependent on capsular thickness and the total surface area of encapsulation. As such, excessive bleb fibrosis and encapsulation of the bleb around the endplate of the drainage device can impair to performance of the drainage device, cause discomfort, and lead to increased intraocular pressure. Complications due to bleb thickness and encapsulation have been reported as great as 40-50% with some drainage devices.

Efforts to prevent or reduce complications due to thick capsules have been made. The drainage devices are made or include materials that deter fibroblast adherence. The antifibrotic agents mitomycin C (MMC) and 5-fluorouracil (5-FU) have been applied intraoperatively and post operatively by placing an agent soaked sponge between the sclera and conjunctival flap during surgery or by injection post-operatively. However, when reviewed by Cochrane database analysis, these treatments have not proven successful at managing complications due to excessive bleb formation and potentially can introduce their own set of complications. Subconjunctival injection of 5-FU can cause corneal epithelial toxicity, which can cause damage ranging from punctate keratopathy to large corneal abrasions. Further, treatment with MMC or 5-FU increases the incidence of conjunctival wound leaks, hypotony, hypotony maculopathy, and suprachoroidal hemorrhage in blebs from trabeculectomy surgery. As such, the use of these co-therapies is typically reserved for high-risk patients and, even then, is used with caution. In short, current methods of reducing excessive bleb formation and bleb encapsulation suffer from at least the aforementioned deficiencies and are ill-suited for most patients.

With the aforementioned deficiencies in mind, described herein are formulations that can contain an amount of a biomolecule or agent, such as an enzyme, that can be effective to generate pores and/or reduce an amount of scar tissue. In embodiments, the formulations can be effective to generate pores and/or reduce an amount of a bleb. In embodiments, the biomolecule or agent, such as an enzyme, can be coupled to a magnetic nanoparticle, which can facilitate localized delivery and/or action of the enzyme. Also provided herein are methods of treating scar tissue, including blebs, which can include the step of delivering a formulation as described herein to or in an area proximate to the scar tissue. In some embodiments, the method can include exposing the scar tissue to a magnetic field. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Methods of Treating Scar Tissue

Provided herein are methods of treating scar tissue, where the methods can contain the step of delivering a formulation as described herein to the scare tissue or an area proximate to the scar tissue. While the description provided herein is presented in the context of treating blebs in subjects undergoing treatment for glaucoma, it will be appreciated that the same formulations and methods described herein can be used to treat other similar types of scar tissue in humans and animals.

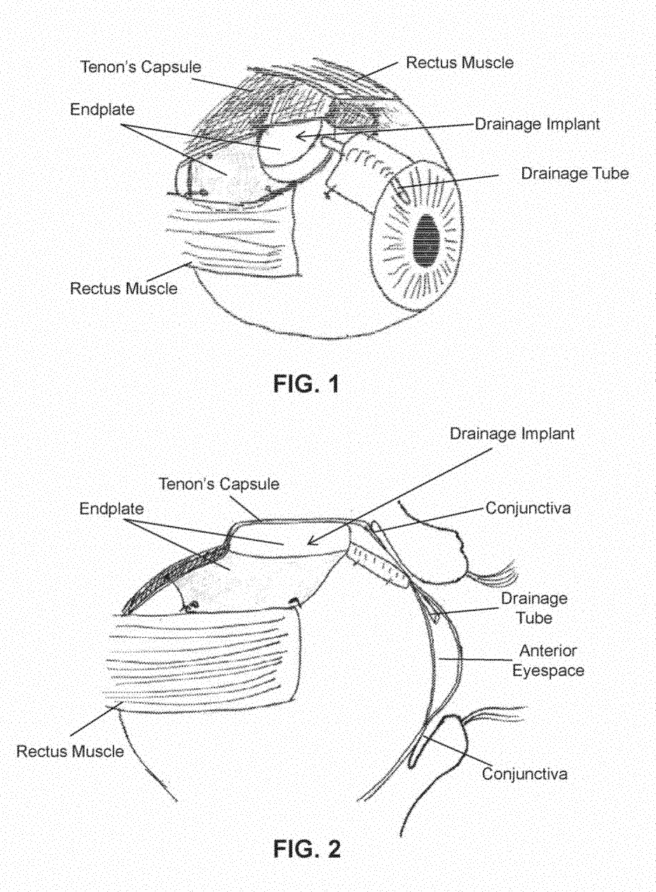

Discussion of the several embodiments of the method begin with FIGS. 1-6, which show placement of a drainage implant in an eye and the subsequent bleb formation over the implant device. As shown in FIGS. 1-2, the implant is placed in the sub-Tenon's capsule region of the eye typically between two rectus muscles. A drainage tube extends from the endplate of the implant into the anterior eyespace. During placement of the drainage device, the Tenon's capsule is stretched over the endplate. At this time, there is little to no fluid present between the Tenon's capsule and the implant. It will also be appreciated that bleb formation is not immediate and is formed as part of the natural healing process that occurs post-operatively.

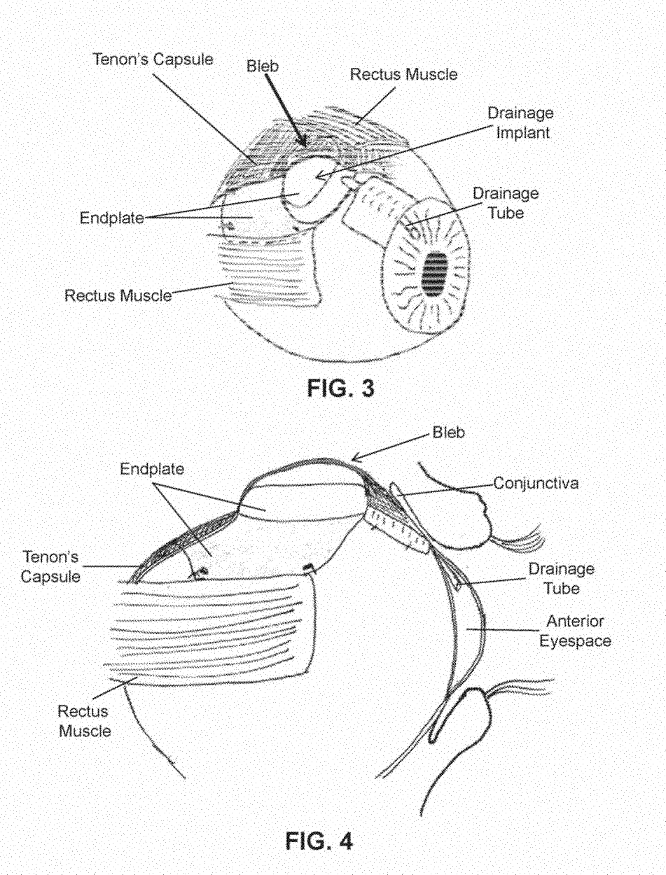

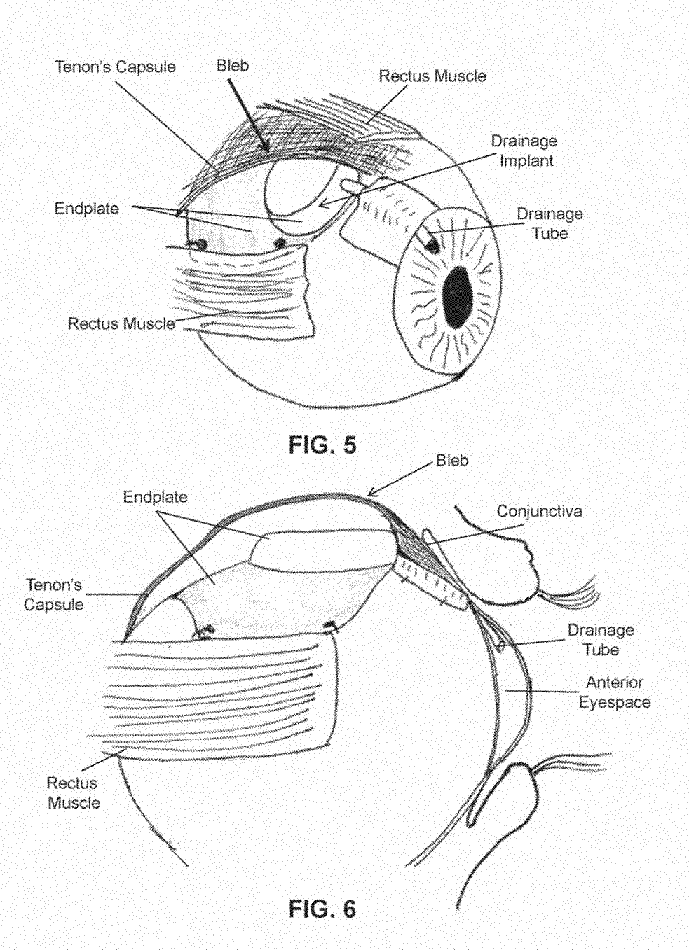

As shown in FIGS. 3-4, the initial bleb tissue forms a fibrous lining of the Tenon's capsule over at least part of the endplate at about 2-3 weeks after drain implantation. Additionally, aqueous fluid of anterior eyespace occurs begins to drain into space between the endplate and the bleb tissue, which causes the bleb to blister. As shown in FIGS. 5-6 a mature bleb forms a fibrous lining of the Tenon's capsule over the endplate at about 3 or more weeks after drain implantation. A functioning bleb forms a blister over the endplate and serves as a reservoir for aqueous fluid from the anterior space. Over time, the fluid in a functioning bleb is diffused through the walls of the bleb and reabsorbed by the body.

As previously discussed, excessive bleb formation can occur and result in a bleb with walls that are too thick or too disorganized to allow movement of the aqueous fluid in the bleb across the wall of the bleb. This can result in a decrease in movement of fluid out of the anterior eye. As such, intraocular pressure can build up as the bleb becomes non-functional. A similar problem can occur with blebs that from as a result of a trabeculectomy procedure.

The methods described herein provide for delivery of enzymes to the bleb wall of a subject, which can generate pores in the bleb wall. These pores can facilitate the movement of fluid from inside the bleb to space outside of the bleb, where it can be reabsorbed by the body. In some embodiments, the enzymes can reduce the thickness of the bleb wall, which can facilitate the movement of fluid from inside the bleb to space outside of the bleb, where it can be reabsorbed by the body. The subject can be a human or any non-human animal, including but not limited to, dog, cat, horse, cow, mouse, rat, or non-human primate.

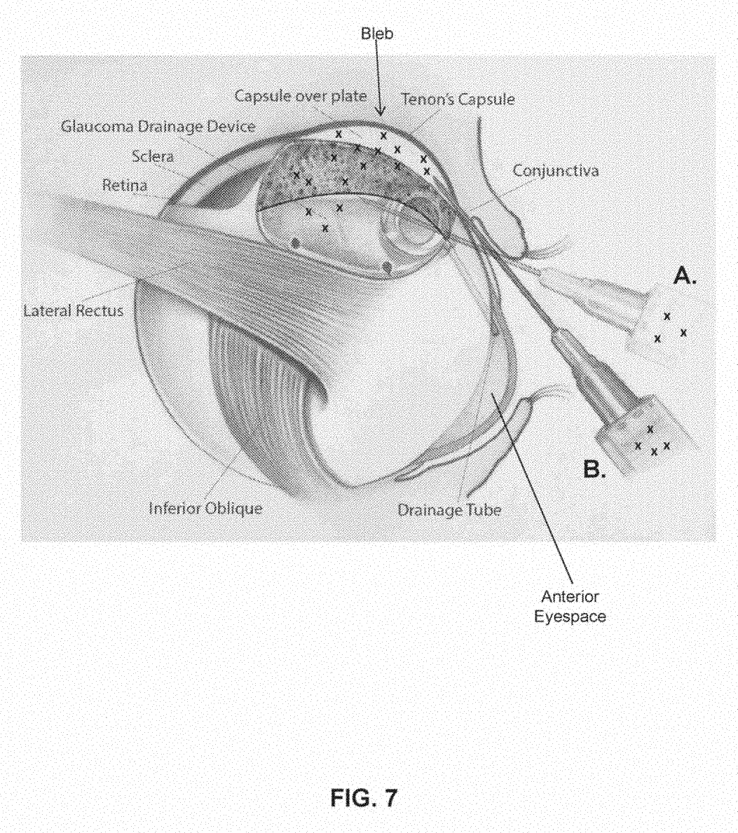

The method of treating a scar tissue in a subject can contain the step of delivering an enzyme or formulation thereof to the scar tissue or a region proximate to the scar tissue. The enzyme or formulation thereof can be delivered by injection. The injection can be, without limitation, intraocular, intravenous, intradermal, intramuscular, subcutaneous, intraventricular, sub-conjunctive, or intralesional. The enzyme or formulation thereof can be delivered topically. The enzyme or formulation thereof can be delivered into target tissues. The scar tissue can be a bleb. As shown in FIG. 7, the enzyme or formulation thereof can be directly injected into the aqueous fluid filling the space between the bleb wall and the implant (needle position (A)). As shown in FIG. 7, the enzyme or formulation thereof can be injected into an area proximate to the bleb capsule, such as into the space immediately outside of the bleb (needle position (B)). In some embodiments, the enzyme or formulation thereof can be injected sub-conjunctively to achieve delivery to an area proximate to the outside of the bleb capsule (see e.g., FIG. 7, needle position B).

The biomolecule(s) or agent(s), such as enzyme(s), that can be contained in the composition or formulation thereof can be FAS-ligand, collagenase, hyaluronidase, serrapeptase, nattokinase, and combinations thereof. In embodiments, the amount of the biomolecule or agent, such as an enzyme, delivered can be at least 1 pg/kg body weight. The concentration of the biomolecule(s) or agent(s), such as enzyme(s), delivered can range from about 0.001 pg/mL or more to 1 mg/mL. The volume of the formulation delivered can be about 0.001, 0.01, 0.1, 1, 2, 3, 4, 5 mL or more. In embodiments, biomolecule(s) or agent(s), such as enzyme(s), or formulation thereof effective for generating pores and/or reducing the amount of scar tissue can be delivered to the scar tissue or area proximate to the scar tissue. In some embodiments, the enzyme can be directly or indirectly (e.g. via a linker) to a magnetic nanoparticle.

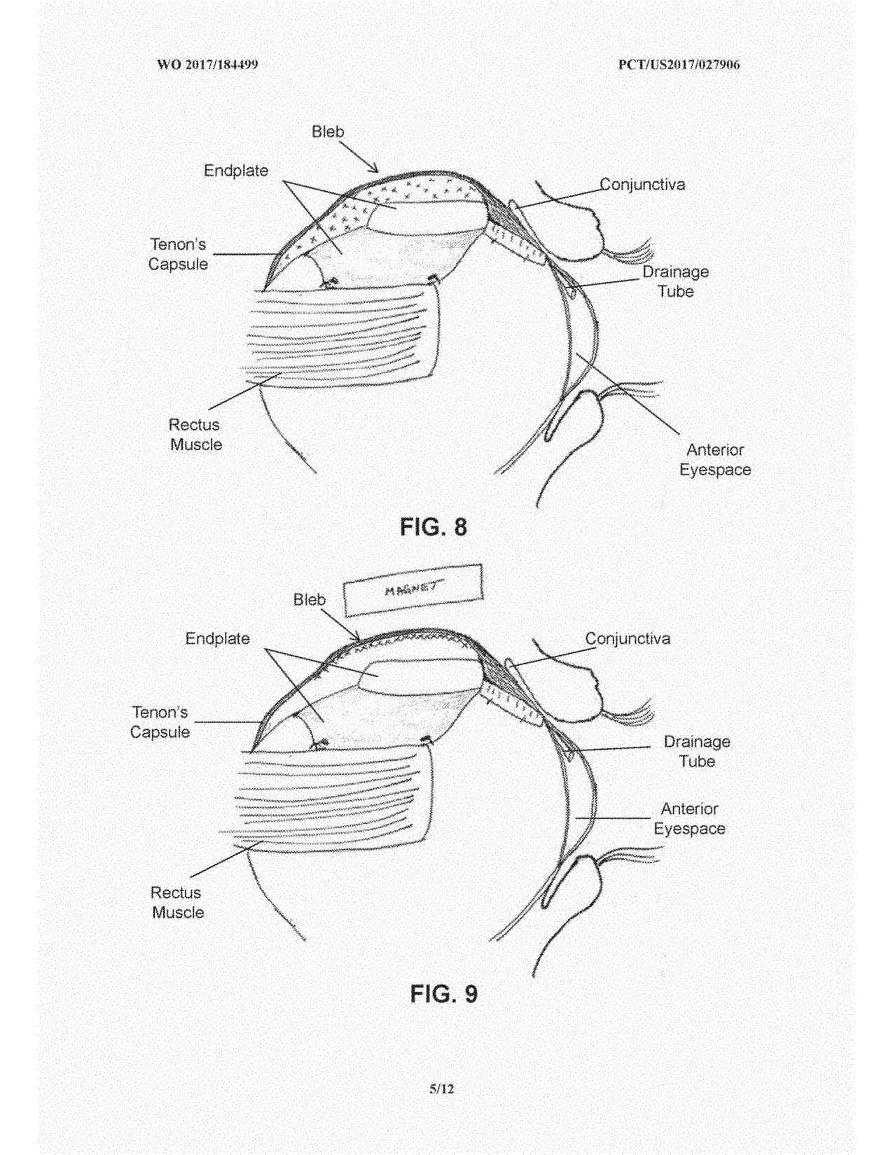

In embodiments where the biomolecule(s) or agent(s), such as enzyme(s), is coupled to a magnetic nanoparticle, the step can further include the step of exposing the scar tissue to a magnetic field. As shown in FIG. 8, when no magnetic field is present, the biomolecule(s) or agent(s), such as enzyme(s), functionalized magnetic nanoparticle can be dispersed throughout the aqueous fluid in the space formed by the bleb wall. As shown in FIG. 9, when a magnetic field is applied to the bleb capsule, the biomolecule(s) or agent(s), such as enzyme(s), functionalized magnetic nanoparticle functionalized magnetic nanoparticles can move towards the magnet until their migration is stopped by the bleb wall. In this way the location of the action of the biomolecule(s) or agent(s), such as enzyme(s), can be controlled and limited to the bleb wall. This can reduce side effects from enzyme action at undesired sites. In action the enzymes can generate pores that extend through the bleb wall and thus can increase the flow of aqueous drainage fluid from the bleb capsule.

In embodiments, the biomolecule(s) or agent(s), such as enzyme(s), or formulation thereof can be co-delivered with a dye that can facilitate visualization of the location of the enzyme or formulation thereof. In some embodiments, the formulation can contain a dye or contrast agent. In other embodiments, the dye or contrast agent can be separate from the formulation until immediately prior to injection. Other dyes and contrast agents will be appreciated by those of skill in the art. The dye or contrast agent can be imaged using a suitable imaging technique sensitive to fluorescence, metal, and/or opacity. Suitable imaging techniques can include, but are not limited to, magnetic resonance imaging (MRI), computed tomography (CT), positron emission tomography (PET) scan, X-ray, or confocal microscopy. In embodiments where the enzyme is coupled to a magnetic particle, imaging of the enzyme within the subject can be obtained using magnetic resonance imaging (MRI) or other metal sensitive technique.

As is also discussed above, the administration of the compositions containing the biomolecule(s) or agent(s), such as enzyme(s), and/or formulations thereof can be administered locally. The compounds and formulations described herein can be administered to the subject in need thereof one or more times per day. In an embodiment, the compound(s) and/or formulation(s) thereof can be administered once daily. In another embodiment, the compound(s) and/or formulation(s) thereof can be administered is administered twice daily. In some embodiments, the compound(s) and/or formulation(s) thereof can be administered 3 or 4 times daily. The compound(s) and/or formulation(s) thereof can be administered one or more times per week, month, or year. In some embodiments the compound(s) and/or formulation(s) thereof can be administered 1 day per week. In other embodiments, the compound(s) and/or formulation(s) thereof can be administered 2 to 7 days per week. The compound(s) and/or formulation(s) thereof can be administered 1, 2, 3, 4, 5, 6, 7, or 8 or more times per month. The compound(s) and/or formulation(s) thereof can be administered 1, 2, 3, 4, 5, 6, 7, 8, 9, 10, 11, 12 times or more per year.

The compositions containing the biomolecule(s) or agent(s), such as enzyme(s), and/or formulations thereof can be administered in a dosage form. The amount or effective amount of the compound(s) and/or formulation(s) thereof can be divided into multiple dosage forms. For example, the effective amount can be split into two dosage forms and the one dosage forms can be administered, for example, in the morning, and the second dosage form can be administered in the evening. Although the effective amount is given over two doses, in one day, the subject receives the effective amount. In some embodiments, the effective amount is about 0.1 to about 1000 mg per day. The effective amount in a dosage form can range from about 0.1 mg/kg to about 1000 mg/kg. The dosage form can be formulated for oral, vaginal, intravenous, transdermal, subcutaneous, intraperitoneal, or intramuscular administration. Preparation of dosage forms for various administration routes are described elsewhere herein.

### Compositions and Pharmaceutical Formulations

Provided herein are compositions and formulations, including pharmaceutical formulations that can contain an enzyme that can break down one or more components of scar tissue or other fibrous tissue. The compositions and formulations described herein can be provided to a subject alone or as an ingredient, such as an active ingredient, in a pharmaceutical formulation or pharmaceutically acceptable salt thereof. Methods of making pharmaceutically acceptable salts are generally known in the art. As such, also described herein are pharmaceutical formulations that can contain one or more of the compositions described herein. In some embodiments, the pharmaceutical formulations can contain an effective amount of a composition described herein. The pharmaceutical formulations can be administered to a subject in need thereof as described elsewhere herein.

### Compositions

Described herein are compositions that can include one or more biomolecules or agents, such as enzymes, that can break down one or more components of scar tissue. In some embodiments, the composition can contain collagenase, hyaluronidase, serrapeptase, nattokinase, FAS-ligand, and combinations thereof. The biomolecules or agents, such as enzymes provided herein, can be used in the preparation of a medicament and/or dosage form that can be effective to break down one or more components of scar tissue and/or treat excessive scar tissue formation, such as bleb formation.

In some embodiments, the composition can contain an amount of collagenase of at least 0.001 pg or more. In some embodiments, the amount of collagenase can be less than 12.5 CDUs. In some embodiments, the amount of collagenase can range from about 0.01 CDU to about 12.5 CDU. The amount of collagenase can range from about 0, 0.01, 0.1, 0.25, 0.5, 0.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 10.25, 10.5, 10.75, 11, 11.25, 11.5, 11.75, 12, 12.25 to 12.5 CDUs.

In embodiments the composition can contain an amount of hyaluronidase ranging from about at least 0.001 pg or more. The amount of hyaluronidase can range from 0 pg to 1 mg or more. The amount of hyaluronidase can range from 0.001 pg to 1 mg or more. The amount of hyaluronidase can range from 0.001 pg, 0.01 pg, 0.1 pg, 1 pg, 10 pg, 100 pg, 1 μg, 10 μg, 100 μg to 1 mg or more.

In embodiments, the composition can contain an amount of FAS-ligand of at least 0.001 pg or more. The amount of FAS-ligand can range from 0 pg to 1 mg or more. The amount of FAS-ligand can range from 0.001 pg to 1 mg or more. The amount of FAS-ligand can range from 0.001 pg, 0.01 pg, 0.1 pg, 1 pg, 10 pg, 100 pg, 1 μg, 10 μg, 100 μg to 1 mg or more.

In embodiments, the composition can contain an amount of nattokinase of at least 0.001 pg or more. The amount of serrapeptase can range from 0 pg to 1 mg or more. The amount of serrapeptase can range from 0.001 pg to 1 mg or more. The amount of serrapeptase can range from 0.001 pg, 0.01 pg, 0.1 pg, 1 pg, 10 pg, 100 pg, 1 μg, 10 μg, 100 μg to 1 mg or more.

In embodiments, the composition can contain an amount of nattokinase of at least 0.001 pg or more. The amount of serrapeptase can range from 0 pg to 1 mg or more. amount of serrapeptase can range from 0.001 pg to 1 mg or more. The amount of serrapeptase can range from 0.001 pg, 0.01 pg, 0.1 pg, 1 pg, 10 pg, 100 pg, 1 μg, 10 μg, 100 μg to 1 mg or more.

In embodiments where the composition contains collagenase and hyaluronidase, the amount of collagenase can range from about 0.01 CDU to 12.5 CDU, and the amount of hyaluronidase can range from about 0.001 pg to about 1 mg or more. In embodiments where the composition contains hyaluronidase and FAS-ligand, the amount of the FAS ligand can range from about 0.001 pg to about 1 mg or more and the amount of hyaluronidase can range from about 0.001 pg to 1 mg or more. In some embodiments where the composition contains collagenase and FAS-ligand, the amount of collagenase can range from about 0.01 CDU to 12.5 CDU and the amount of FAS-ligand can range from about 0.001 pg to about 1 mg or more. In embodiments where the composition contains hyaluronidase, collagenase, and FAS-ligand, the amount of hyaluronidase can range from about 0.001 pg to about 1 mg or more, the amount of collagenase can range from about 0.01 CDU to 12.5 CDU, and the amount of FAS-ligand can range from about 0.001 pg to 1 mg or more.

The enzyme(s) can be coupled to magnetic nanoparticles to form an enzyme functionalized magnetic nanoparticle. The enzyme can be directly coupled to the magnetic nanoparticle. The enzyme can be indirectly coupled to (e.g. via a linker) to the magnetic nanoparticle. The magnetic nanoparticle can be ferrite nanoparticles. Ferrite nanoparticles can have the crystal structure of maghemite or magnetite. The magnetic nanoparticles can be metallic nanoparticles. In some embodiments, the nanoparticles can include iron or cobalt. The surface of the nanoparticles can be modified by one or more other chemicals, elements, or compounds, including but not limited to, surfactants, silica, silicones, phosphoric acid derivatives, and dyes. The nanoparticles can be passivated by gentle oxidation, surfactants, polymers, or precious metals. The magnetic nanoparticles can have a core-shell anatomy. Other suitable magnetic nanoparticles will be appreciated by those of skill in the art.

The magnetic nanoparticles can be smaller than 1 micrometer in diameter. The magnetic nanoparticles can range from about 1 to 1000 nanometers in diameter. In some embodiments, the magnetic nanoparticles can form magnetic nanoparticle clusters, which can include a population of magnetic nanoparticles. These clusters, which can also be called magnetic nanobeads, can have a diameter ranging from about 50 to 200 nanometers.

Methods of synthesizing magnetic nanoparticles are known to those of skill in the art. Methods of coupling enzymes, directly or indirectly, are generally known in the art.

### Pharmaceutical Formulations

Also described herein are pharmaceutical formulations containing an amount of a composition as described herein. The amount can be an amount effective to generate pores in a bleb wall or other scar tissue and/or reduce the amount of bleb wall or other scar tissue. Pharmaceutical formulations can be formulated for delivery via a variety of routes and can contain a pharmaceutically acceptable carrier. Techniques and formulations generally can be found in Remmington's Pharmaceutical Sciences, Meade Publishing Co., Easton, Pa. (20^{th} Ed., 2000), the entire disclosure of which is herein incorporated by reference. For systemic administration, an injection is useful, including intramuscular, intravenous, intraperitoneal, and subcutaneous. For injection, the therapeutic compositions of the invention can be formulated in liquid solutions, for example in physiologically compatible buffers such as Hank's solution or Ringer's solution. In addition, the therapeutic compositions can be formulated in solid form and redissolved or suspended immediately prior to use. Lyophilized forms are also included. Pharmaceutical compositions of the present invention are characterized as being at least sterile and pyrogen-free. These pharmaceutical formulations include formulations for human and veterinary use.

Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxyl methylcellulose, and polyvinyl pyrrolidone, which do not deleteriously react with the active composition.

The pharmaceutical formulations can be sterilized, and if desired, mixed with auxiliary agents, such as lubricants, preservatives, stabilizers, wetting agents, emulsifiers, salts for influencing osmotic pressure, buffers, coloring, flavoring and/or aromatic substances, and the like, which do not deleteriously react with the active composition.

The pharmaceutical formulations can be administered to a subject in need thereof. The subject in need thereof can have a disease, disorder, or a symptom thereof. Example disease or disorder can include, but are not limited to, glaucoma. In some embodiments, the subject in need thereof has a bleb or other scar tissue.

The pharmaceutical formulation can be formulated to be compatible with its intended route of administration. Examples of routes of administration include intraocular, sub-conjunctival, parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical formulations suitable for injectable use can include sterile aqueous solutions (where water soluble) or dispersions and sterile powders for the extemporaneous preparation of sterile injectable solutions or dispersions. For intravenous administration, suitable carriers can include physiological saline, bacteriostatic water, Cremophor EM™ (BASF, Parsippany, N.J.) or phosphate buffered saline (PBS). Injectable pharmaceutical formulations can be sterile and can be fluid to the extent that easy syringability exists. Injectable pharmaceutical formulations can be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it can be useful to include isotonic agents, for example, sugars, polyalcohols such as mannitol, sorbitol, sodium chloride in the composition. Prolonged absorption of injectable compositions can be brought about by incorporating an agent which delays absorption, for example, aluminum monostearate and gelatin.

Sterile injectable solutions can be prepared by incorporating any of the compositions as described herein in an amount in an appropriate solvent with one or a combination of ingredients enumerated herein, as required, followed by filtered sterilization. Generally, dispersions can be prepared by incorporating the nucleic acid vectors into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional desired ingredient from a previously sterile-filtered solution thereof.

Administration can also be by transmucosal or transdermal means. For transmucosal or transdermal administration, penetrants appropriate to the barrier to be permeated can be used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, any of the compositions described herein can be formulated into ointments, salves, gels, or creams as generally known in the art. In some embodiments, the composition can be applied via transdermal delivery systems, which can slowly release the composition for percutaneous absorption. Permeation enhancers can be used to facilitate transdermal penetration of the active factors in the conditioned media. Transdermal patches are described in for example, U.S. Pat. No. 5,407,713; 5,352,456; 5,332,213; 5,336,168; 5,290,561; 5,254,346; 5,164,189; 5,163,899; 5,088,977; 5,087,240; 5,008,110; and 4,921,475.

Administration of the composition(s) is not restricted to a single route, but may encompass administration by multiple routes. For instance, exemplary administrations by multiple routes include, among others, a combination of intradermal and intramuscular administration, or intradermal and subcutaneous administration. Multiple administrations may be sequential or concurrent. Other modes of application by multiple routes will be apparent to the skilled artisan.

The pharmaceutical formulations can be administered to a subject by any suitable method that allows the agent to exert its effect on the subject in vivo. For example, the formulations or other compositions described herein can be administered to the subject by known procedures including, but not limited to, by oral administration, sublingual or buccal administration, parenteral administration, transdermal administration, via inhalation, via nasal delivery, vaginally, rectally, and intramuscularly. The formulations or other compositions described herein can be administered parenterally, by epifascial, intracapsular, intracutaneous, subcutaneous, ocular, intradermal, intrathecal, intramuscular, intraperitoneal, intrasternal, intravascular, intravenous, parenchymatous, and/or sublingual delivery. Delivery can be by injection, infusion, catheter delivery, or some other means, such as by tablet or spray. In some embodiments, the composition can be administered to the subject by way of delivery directly to the heart tissue, such as by way of a catheter inserted into, or in the proximity of the subjects heart, or by using delivery vehicles capable of targeting the drug to the heart. For example, the compositions described herein can be conjugated to or administered in conjunction with an agent that is targeted to the eye, such as an aptamer, antibody or antibody fragment. The compositions described herein can be administered to the subject by way of delivery directly to the tissue of interest, such as by way of a catheter inserted into, or in the proximity of the subject's tissue of interest, or by using delivery vehicles capable of targeting the compositions to the scar tissue or eye, such as an antibody or antibody fragment.

For oral administration, a formulation as described herein can be presented as capsules, tablets, powders, granules, or as a suspension or solution. The formulation can contain conventional additives, such as lactose, mannitol, cornstarch or potato starch, binders, crystalline cellulose, cellulose derivatives, acacia, cornstarch, gelatins, disintegrators, potato starch, sodium carboxymethylcellulose, dibasic calcium phosphate, anhydrous or sodium starch glycolate, lubricants, and/or or magnesium stearate.

For parenteral administration (i.e., administration by through a route other than the alimentary canal), the formulations described herein can be combined with a sterile aqueous solution that is isotonic with the blood of the subject. Such a formulation can be prepared by dissolving the active ingredient in water containing physiologically-compatible substances, such as sodium chloride, glycine and the like, and having a buffered pH compatible with physiological conditions, so as to produce an aqueous solution, then rendering the solution sterile. The formulation can be presented in unit or multi-dose containers, such as sealed ampoules or vials. The formulation can be delivered by injection, infusion, or other means known in the art.

For transdermal administration, the formulation described herein can be combined with skin penetration enhancers, such as propylene glycol, polyethylene glycol, isopropanol, ethanol, oleic acid, N-methylpyrrolidone and the like, which increase the permeability of the skin to the nucleic acid vectors of the invention and permit the nucleic acid vectors to penetrate through the skin and into the bloodstream. The formulations and/or compositions described herein can be further combined with a polymeric substance, such as ethylcellulose, hydroxypropyl cellulose, ethylene/vinylacetate, polyvinyl pyrrolidone, and the like, to provide the composition in gel form, which can be dissolved in a solvent, such as methylene chloride, evaporated to the desired viscosity and then applied to backing material to provide a patch.

The formulation can contain an effective amount of an enzyme or combination of enzymes. Effective amounts of the enzyme(s) are described in relation to the compositions and methods.

### Combination Therapy

The pharmaceutical formulations or other compositions described herein can include or can be administered to a subject either as a single agent, or in combination with one or more other agents. Additional agents include but are not limited to DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, imaging dyes, contrast agents, and chemotherapeutics. Multiple compositions as described herein can be administered simultaneously in a combination treatment.

Suitable antipyretics include, but are not limited to, non-steroidal anti-inflammants (e.g. ibuprofen, naproxen, ketoprofen, and nimesulide), aspirin and related salicylates (e.g. choline salicylate, magnesium salicylae, and sodium salicaylate), paracetamol/acetaminophen, metamizole, nabumetone, phenazone, and quinine.

Suitable anxiolytics include, but are not limited to, benzodiazepines (e.g. alprazolam, bromazepam, chlordiazepoxide, clonazepam, clorazepate, diazepam, flurazepam, lorazepam, oxazepam, temazepam, triazolam, and tofisopam), serotenergic antidepressants (e.g. selective serotonin reuptake inhibitors, tricyclic antidepresents, and monoamine oxidase inhibitors), mebicar, afobazole, selank, bromantane, emoxypine, azapirones, barbituates, hyxdroxyzine, pregabalin, validol, and beta blockers.

Suitable antipsychotics include, but are not limited to, benperidol, bromoperidol, droperidol, haloperidol, moperone, pipaperone, timiperone, fluspirilene, penfluridol, pimozide, acepromazine, chlorpromazine, cyamemazine, dizyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, tiotixene, zuclopenthixol, clotiapine, loxapine, prothipendyl, carpipramine, clocapramine, molindone, mosapramine, sulpiride, veralipride, amisulpride, amoxapine, aripiprazole, asenapine, clozapine, blonanserin, iloperidone, lurasidone, melperone, nemonapride, olanzaprine, paliperidone, perospirone, quetiapine, remoxipride, risperidone, sertindole, trimipramine, ziprasidone, zotepine, alstonie, befeprunox, bitopertin, brexpiprazole, cannabidiol, cariprazine, pimavanserin, pomaglumetad methionil, vabicaserin, xanomeline, and zicronapine.

Suitable analgesics include, but are not limited to, paracetamol/acetaminophen, non-steroidal anti-inflammants (e.g. ibuprofen, naproxen, ketoprofen, and nimesulide), COX-2 inhibitors (e.g. rofecoxib, celecoxib, and etoricoxib), opioids (e.g. morphine, codeine, oxycodone, hydrocodone, dihydromorphine, pethidine, buprenorphine), tramadol, norepinephrine, flupiretine, nefopam, orphenadrine, pregabalin, gabapentin, cyclobenzaprine, scopolamine, methadone, ketobemidone, piritramide, and aspirin and related salicylates (e.g. choline salicylate, magnesium salicylae, and sodium salicaylate).

Suitable antispasmodics include, but are not limited to, mebeverine, papverine, cyclobenzaprine, carisoprodol, orphenadrine, tizanidine, metaxalone, methodcarbamol, chlorzoxazone, baclofen, dantrolene, baclofen, tizanidine, and dantrolene.

Suitable anti-inflammatories include, but are not limited to, prednisone, non-steroidal anti-inflammants (e.g. ibuprofen, naproxen, ketoprofen, and nimesulide), COX-2 inhibitors (e.g. rofecoxib, celecoxib, and etoricoxib), and immune selective anti-inflammatory derivatives (e.g. submandibular gland peptide-T and its derivatives).

Suitable anti-histamines include, but are not limited to, H₁-receptor antagonists (e.g. acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbromapheniramine, dexchlorpheniramine, dimenhydrinate, dimetindene, diphenhydramine, doxylamine, ebasine, embramine, fexofenadine, hydroxyzine, levocetirzine, loratadine, meclozine, mirtazapine, olopatadine, orphenadrine, phenindamine, pheniramine, phenyltoloxamine, promethazine, pyrilamine, quetiapine, rupatadine, tripelennamine, and triprolidine), H₂-receptor antagonists (e.g. cimetidine, famotidine, lafutidine, nizatidine, rafitidine, and roxatidine), tritoqualine, catechin, cromoglicate, nedocromil, and β2-adrenergic agonists.

Suitable anti-infectives include, but are not limited to, amebicides (e.g. nitazoxanide, paromomycin, metronidazole, tnidazole, chloroquine, and iodoquinol), aminoglycosides (e.g. paromomycin, tobramycin, gentamicin, amikacin, kanamycin, and neomycin), anthelmintics (e.g. pyrantel, mebendazole, ivermectin, praziquantel, abendazole, miltefosine, thiabendazole, oxamniquine), antifungals (e.g. azole antifungals (e.g. itraconazole, fluconazole, posaconazole, ketoconazole, clotrimazole, miconazole, and voriconazole), echinocandins (e.g. caspofungin, anidulafungin, and micafungin), griseofulvin, terbinafine, flucytosine, and polyenes (e.g. nystatin, and amphotericin b), antimalarial agents (e.g. pyrimethamine/sulfadoxine, artemether/lumefantrine, atovaquone/proquanil, quinine, hydroxychloroquine, mefloquine, chloroquine, doxycycline, pyrimethamine, and halofantrine), antituberculosis agents (e.g. aminosalicylates (e.g. aminosalicylic acid), isoniazid/rifampin, isoniazid/pyrazinamide/rifampin, bedaquiline, isoniazid, ethanmbutol, rifampin, rifabutin, rifapentine, capreomycin, and cycloserine), antivirals (e.g. amantadine, rimantadine, abacavir/lamivudine, emtricitabine/tenofovir, cobicistat/elvitegravir/emtricitabine/tenofovir, efavirenz/emtricitabine/tenofovir, avacavir/lamivudine/zidovudine, lamivudine/zidovudine, emtricitabine/tenofovir, emtricitabine/opinavir/ritonavir/tenofovir, interferon alfa-2v/ribavirin, peginterferon alfa-2b, maraviroc, raltegravir, dolutegravir, enfuvirtide, foscarnet, fomivirsen, oseltamivir, zanamivir, nevirapine, efavirenz, etravirine, rilpiviirine, delaviridine, nevirapine, entecavir, lamivudine, adefovir, sofosbuvir, didanosine, tenofovir, avacivr, zidovudine, stavudine, emtricitabine, xalcitabine, telbivudine, simeprevir, boceprevir, telaprevir, lopinavir/ritonavir, fosamprenvir, dranuavir, ritonavir, tipranavir, atazanavir, nelfinavir, amprenavir, indinavir, sawuinavir, ribavirin, valcyclovir, acyclovir, famciclovir, ganciclovir, and valganciclovir), carbapenems (e.g. doripenem, meropenem, ertapenem, and cilastatin/imipenem), cephalosporins (e.g. cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, and ceftazidime), glycopeptide antibiotics (e.g. vancomycin, dalbavancin, oritavancin, and telvancin), glycylcyclines (e.g. tigecycline), leprostatics (e.g. clofazimine and thalidomide), lincomycin and derivatives thereof (e.g. clindamycin and lincomycin), macrolides and derivatives thereof (e.g. telithromycin, fidaxomicin, erthromycin, azithromycin, clarithromycin, dirithromycin, and troleandomycin), linezolid, sulfamethoxazole/trimethoprim, rifaximin, chloramphenicol, fosfomycin, metronidazole, aztreonam, bacitracin, penicillins (amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxaxillin, dicloxacillin, and nafcillin), quinolones (e.g. lomefloxacin, norfloxacin, ofloxacin, qatifloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, and sparfloxacin), sulfonamides (e.g. sulfamethoxazole/trimethoprim, sulfasalazine, and sulfasoxazole), tetracyclines (e.g. doxycycline, demeclocycline, minocycline, doxycycline/salicyclic acid, doxycycline/omega-3 polyunsaturated fatty acids, and tetracycline), and urinary anti-infectives (e.g. nitrofurantoin, methenamine, fosfomycin, cinoxacin, nalidixic acid, trimethoprim, and methylene blue).

Suitable chemotherapeutics include, but are not limited to, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, cytoxan, cyclophosphamide, decitabine, dexamethasone, docetaxel, hydroxyurea, decarbazine, leuprolide, epirubicin, oxaliplatin, asparaginase, estramustine, cetuximab, vismodegib, aspargainase erwinia chyrsanthemi, amifostine, etoposide, flutamide, toremifene, fulvestrant, letrozole, degarelix, pralatrexate, methotrexate, floxuridine, obinutuzumab, gemcitabine, afatinib, imatinib mesylatem, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octretide, dasatinib, regorafenib, histrelin, sunitinib, siltuximab, omacetaxine, thioguanine (tioguanine), dabrafenib, erlotinib, bexarotene, temozolomide, thiotepa, thalidomide, BCG, temsirolimus, bendamustine hydrochloride, triptorelin, aresnic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, and all-trans retinoic acid.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### Example 1

The ability of collagenase, hyaluronidase, and FAS ligand to augment or otherwise alter the permeability through a capsule formed over a drainage implant plate was evaluated. An in vivo rat model was used in which implants were implanted sub-dermally on the back of rats. The implants also contained a small silicone tube attached to the implant that could be accessed through the skin. In the model, a fibrous capsule naturally forms over the implant in reaction to the foreign material. The capsules were allowed to mature for about 6-10 weeks. Injections (about 0.1 mL total volume) were then administered into the encapsulated implant area. The injections contained saline (a control). FAS-ligand (about 0.01mg), or collagenase (138 CDU, 50 CDU, or 12.5 CDU). Two days after injection, the implants were isolated and the permeability of the capsule was measured using a Harvard pump system by running fluid at 3 μL/min down the silicone tube that was connected to the implant. Tissues were preserved for histological analysis.

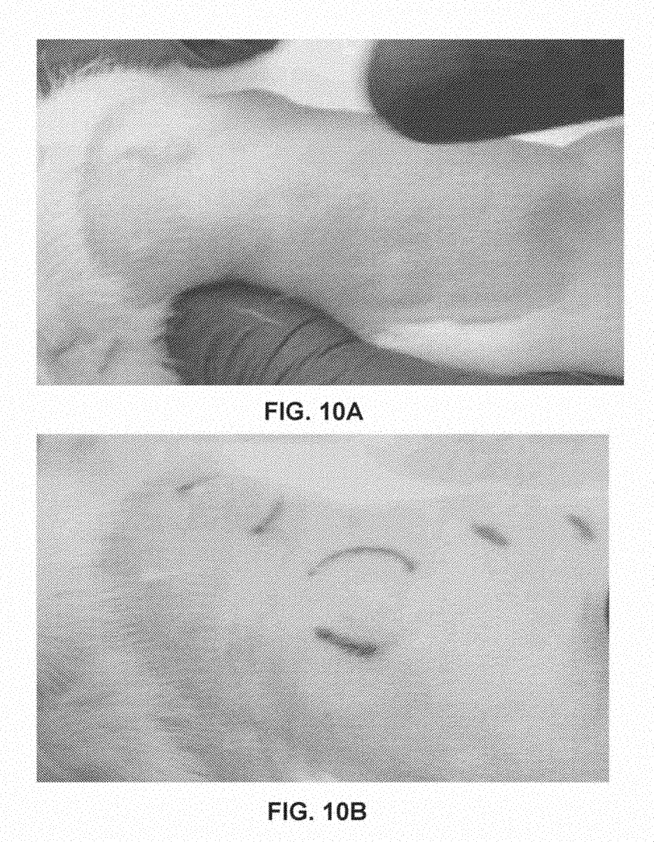

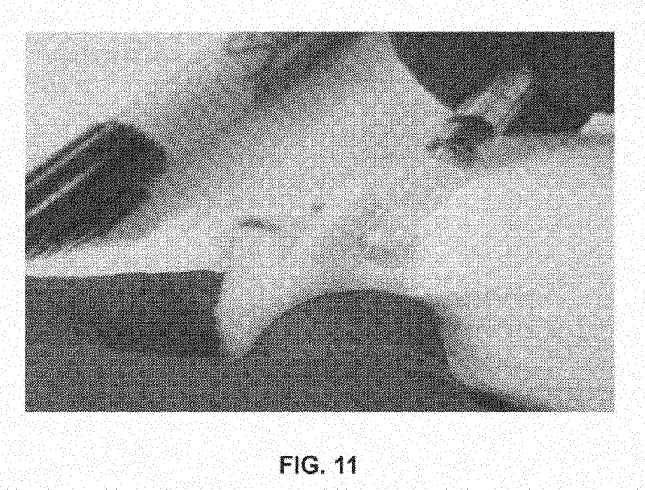

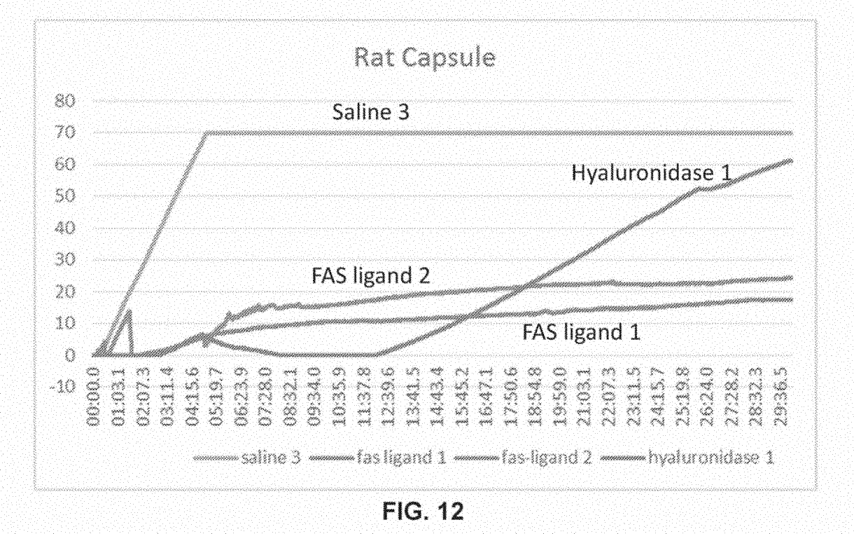

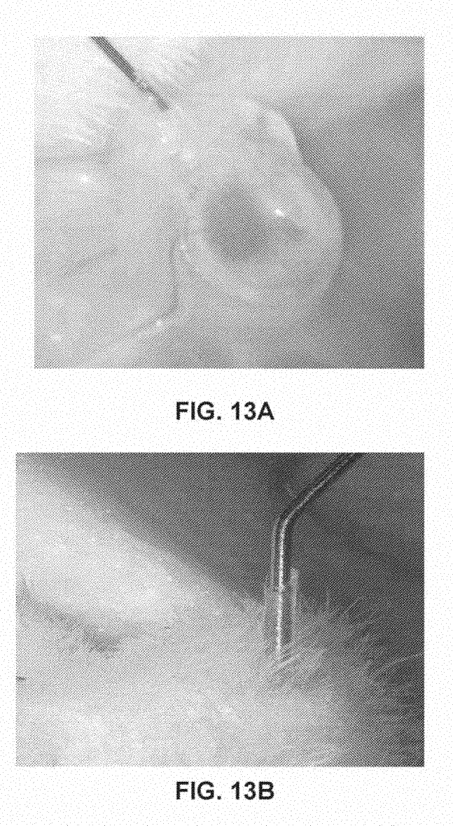

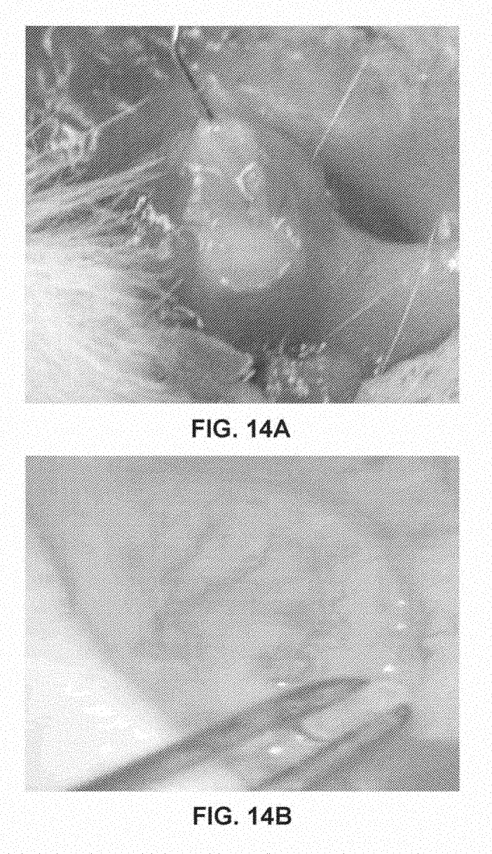

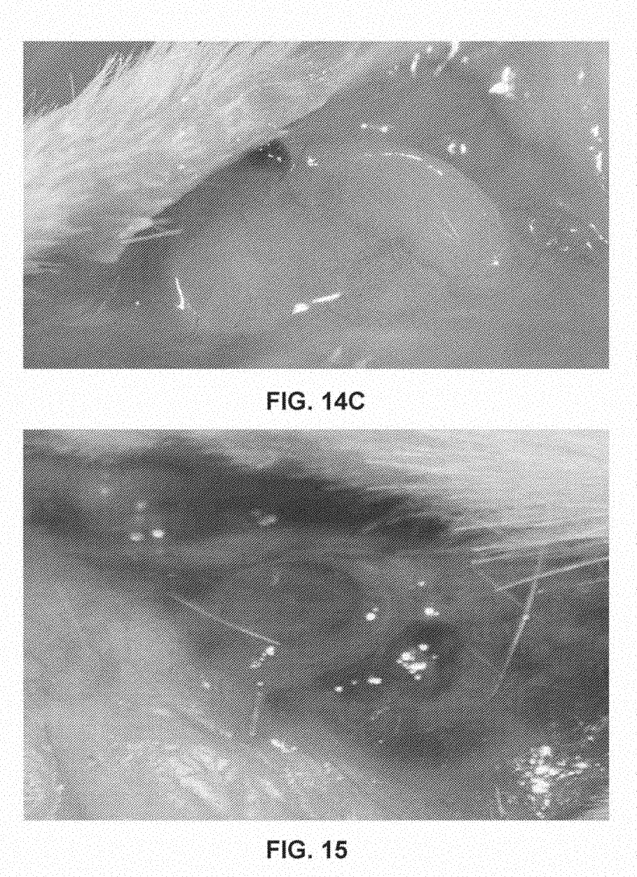

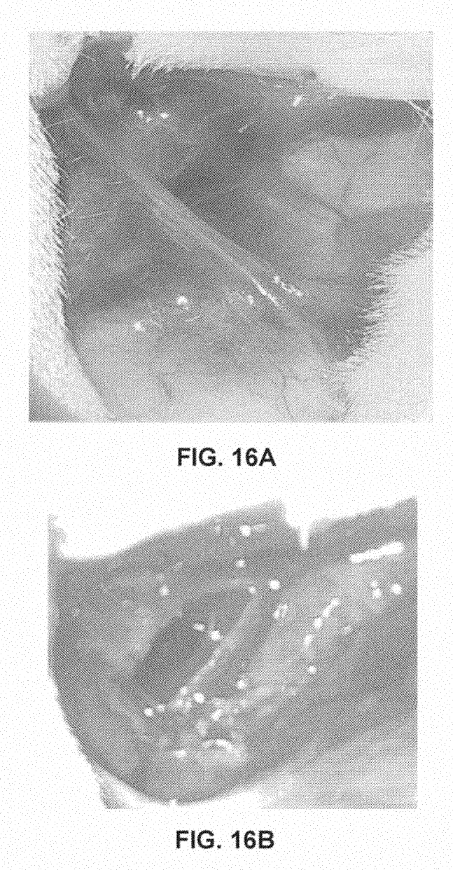

The results are demonstrated in FIGS. 10A-16B. The results suggest that at least FAS-ligand can be effective at increasing the permeability of the capsule while maintaining normal-appearing structural integrity of the capsule.

1. A formulation for treating excessive bleb formation in a subject, the formulation comprising:
an enzyme in an amount effective to form pores in a bleb, where the bleb is formed as the result of a surgical procedure to treat glaucoma in the subject.

2. The formulation of claim 1, wherein the surgical procedure is implantation of an ocular drainage device.

3. The formulation of claim 1, wherein the surgical procedure is a trabeculectomy.

4. The formulation of claim 1, wherein the enzyme is coupled to a magnetic nanoparticle.

5. The formulation of claim 1, wherein the enzyme is selected from the group consisting of: collagenase, hyaluronidase, serrapeptase, nattokinase, FAS-ligand and combinations thereof.

6. The formulation of 1, wherein the formulation is formulated for ocular injection.

7. A formulation for treating scar tissue in a subject, the formulation comprising:
an agent in an amount effective to form pores in the scar tissue or reduce an amount of scar tissue.

8. The formulation of claim 7, wherein the agent is an enzyme.

9. The formulation of claim 1, wherein the agent is selected from the group consisting of: collagenase, hyaluronidase, serrapeptase, nattokinase, FAS-ligand, and combinations thereof.

10. The formulation of claim 1, wherein the agent is coupled to a magnetic nanoparticle.

11. A method of treating a scar tissue in a subject, the method comprising:
delivering an agent or formulation thereof to the scar tissue or a region proximate to the scar tissue.

12. The method of claim 11, wherein the agent is an enzyme.

13. The method of claim 11, wherein the agent is selected from the group consisting of: collagenase, hyaluronidase, serrapeptase, nattokinase, FAS-ligand and combinations thereof.

14. The method of claim 11, wherein the agent is coupled to a magnetic nanoparticle.

15. The method of claim 14, further comprising the step of exposing the scar tissue to a magnetic field.

16. The method of claim 11, wherein the subject is a human.

17. The method of claim 11, wherein the subject is a non-human animal.

18. The method of claim 11, wherein the scar tissue is a bleb.

19. The method of claim 11, further comprising the step of co-delivering a dye with the enzyme or formulation thereof.

20. The method of claim 11, further comprising the step of imaging the subject using resonance imaging (MRI), computed tomography (CT), positron emission tomography (PET) scan, X-ray, or confocal microscopy.
